(19) United States
(12) Patent Application Publication
(10) Pub. No.: US 2015/0202372 A1
(43) Pub. Date: Jul. 23, 2015

(54) SYRINGE FOR THE APPLICATION OF MULTIPLE TOPICAL TREATMENTS

(71) Applicants: Eram Ali, Midland, TX (US); Gary Schwede, Lubbock, TX (US); Jeremy Kimbrough, La Jolla, CA (US); Rhett Goodman, College Station, TX (US); Zach Franklin, College Station, TX (US); Michael Croft, College Station, TX (US)

(72) Inventors: Eram Ali, Midland, TX (US); Gary Schwede, Lubbock, TX (US); Jeremy Kimbrough, La Jolla, CA (US); Rhett Goodman, College Station, TX (US); Zach Franklin, College Station, TX (US); Michael Croft, College Station, TX (US)

(21) Appl. No.: 14/159,839

(22) Filed: Jan. 21, 2014

(51) Int. Cl. A61M 5/315

(52) U.S. Cl. CPC A61M 5/315

(57) ABSTRACT

A syringe comprising: an applicator with a tip; a chamber that is in fluid communication with the application tip; a reservoir containing housing at least one ampule of medication; said reservoir separated by a port; a press that is configured be received by the reservoir and push the ampules of medication into the chamber through the port; and a plunger for pushing the ampules' contents of medication through the application tip. In one mode of operation: ampules of medication (each ampule can contain a different medication) are deposited into the reservoir of the syringe; the press is inserted into the reservoir and manipulated to push the ampules through the port into the chamber; and, the plunger is depressed to force the medications through the application tip. Preferably the syringe is used for dispensing topical medication, but it can be adapted for other medical and non-medical uses.

### CROSS-REFERENCE TO RELATED APPLICATIONS

Not applicable.

### STATEMENT REGARDING FEDERALLY SPONSORED RESEARCH OR DEVELOPMENT

Not applicable.

### BACKGROUND OF INVENTION

1. Field of the Invention

The present application relates to the field of medical devices.

2. Background of the Invention

Veterinarians often apply topical medications to the skin of animals to treat ailments such as bacterial or fungal infections and to reduce pruritis (itchiness) or inflammation. Typical topical medications include antifungal, antibacterial, antiparasitic or steroidal ointments, gels and creams. Frequently, animals need multiple medications and, in those cases, a veterinarian may topically administer those medications immediately, one-after-the-other.

Topically administering multiple medications to animals, one-after-the-other, can be problematic in some circumstances. For instance, certain topical medications applied to “sensitive areas” such as the ear canal can agitate the animal, cause additional discomfort, so the animal may resist application of any further medication, thereby reducing therapeutic options. Animal resistance can result in the veterinarian or veterinary technician being injured by the animal or in the misapplication of the medications. Even when multiple medications are properly applied to an animal without injury to the veterinarian, the administration of multiple medications, one after the other, is tedious and time consuming. It goes without saying that a need exists for an apparatus that enables veterinarians to administer more than one topical treatment to an animal with a single application.

In view of the above need, multiple medication or multi-formula treatments are known wherein medications that are commonly applied at the same time are mixed in a single application. Sometimes, such mixes are known as “two-in-one” or even a “three-in-one” ointments. Although useful in many situations, multi-formula treatments are problematic because the mixes are available in pre-set dosages of each medication and many real life circumstances require medication dosages that are different than said pre-set dosages. An animal may be allergic or sensitive to one of the ingredients in a “three-in-one” ointment. For example, the drug gentamicin is an antibiotic that veterinarians frequently prescribe to treat ear infections. However, gentamicin is highly nephrotoxic, meaning it cannot be prescribed to a patient with a history of kidney problems. A doctor would be unable to prescribe a three-in-one containing gentamicin to a patient with a history of kidney disease, even if the patient requires the other two drugs in the three-in-one formula. Thus, a need exits for apparatus and related methods for mixing multi-medication topical treatments customizable formulas and dosages depending on the needs of the patient.

Even mixes that contain commonly used combinations of medications can feature disparate medications that will react with each other adversely. That is to say, chemical reactions within the mixes can reduce the efficacy of at least one of the medications when multiple medications are stored together for an extended period of time. As a result, there exists a need to be able to store separate medications and then mix them immediately prior to use.

There are examples of devices that store liquids or gels separately and allow them to be mixed upon use. The most basic of these examples are containers that feature two or more chambers for chemicals that do not mix until being dispensed from the containers. For example, U.S. Pat. No. 1,363,064 (1920) to Stegath, et al. and U.S. Pat. No. 3,166,221 (1965) to Nielson teaches a collapsible tube (e.g., a toothpaste tube) with two chambers. The disclosed tube is essentially constructed of two side-by-side chambers. The tube has two adjacent openings for each chamber so that contents of each chamber can be directed to a target at the same time. U.S. Pat. No. 1,894,115 (1933) to Murphy, et al. teaches a similar tube, but with a single nozzle. Containers such as these are frequently used in wide variety common products, for example: toothpaste containing baking soda and peroxide; and dual-force foaming drain opener. These containers can be useful in many situations, but are nevertheless limited in dosage customization and by the number of chemicals that can be mixed in one application.

Multi-chambered syringes have also been developed for applying multiple medications at once. The most common types of multi-chambered syringes feature two chambers side by side. Each chamber is typically filled with disparate medications that cannot be stored together. When a user depresses a plunger, the plunger pushes both medicines in the two chambers into a mixing area, where the medications are mixed together. Further depressing the plunger forces the liquid in the mixing area through needle for hypodermic injection into a patient. This basic model is described in several patents. See U.S. Pat. No. 2,168,686 (1934) Saffir, U.S. Pat. No. 3,572,336 (1971) to Hershberg, and U.S. Pat. No. 6,612,465 to Pierson (2003). Dual chambered syringes are not versatile. Using a single medication requires the dose to be halved so that one chamber is not empty prior to application so that deadly air bubbles are avoided. Also, it cannot be easily adapted to be used with more than two medications because the devices only feature two chambers.

There are variations to the basic model of two side by side chambers containing medications that are mixed by pushing a plunger and then injected hypodermically. U.S. Pat. No. 553,234 (1896) to Finot teaches a syringe with two chambers where the first chamber is located inside of the second chamber. U.S. Pat. No. 4,915,695 (1990) to Koobs teaches a syringe with three chambers instead of two. U.S. Pat. No. 7,753,890 (2006) to Thorne, et al, discloses a syringe with two chambers where the syringe will inject a first medication to a patient and then a second medication without mixing the medications first. These variations have some of the same limitations as the basic model: they cannot be easily adapted for fewer or greater medications than the number of chambers in the syringe.

Another variation of the basic model is disclosed in U.S. Pat. No. 4,581,015 (1984) to Alfano. This patent teaches a syringe with a side port so that additional medication can be introduced to the syringe. This syringe can be used with one medication or multiple medications. However, this model is not easy to use and requires additional syringes to dispense more than one medication.

Another variation to the basic model includes U.S. Pat. App. No. 2008/0177223 to Johnston. This patent teaches a syringe that is designed to inject multiple vaccines into baby birds with pipe-like compartments connected to a single plunger apparatus. This syringe is complex, not user friendly and cannot easily be adapted to other purposes.

U.S. Pat. App. No. 2011/0021905 teaches a high tech version of the basic model. Two or more vials of a medication can be inserted into an apparatus. The apparatus is connected to a hypodermic needle. A computer is connected to both the needle and vials and dosages are controlled electronically. This allows flexibility in dosages and number of medications, but the device is complex and has the potential to be an overly expensive product that performs a function that may be accomplished by a less expensive syringe.

Additionally, there several variations to the dual-chambered syringe that do not include a hypodermic needle. U.S. Pat. No. 5,819,988 (1998) to Sawhney, et al. teaches a syringe that uses two chambers and a plunger to dispense gels. U.S. Pat. No. 6,613,021 (2003) to Sogaro teaches a multi-chamber syringe-like packaging for dispensing multiple components that harden upon mixing. These syringes have the same problems as previously mentioned inventions: they lack flexibility to change the number of items mixed.

Another variation to a dual chambered medicine dispenser is U.S. Pat. No. 3,269,389 to Meurer et al. This patent teaches a two chambered spray-on medication for noses and throats. When the two chambers are squeezed, the contents of the chambers are mixed in a compartment and dispersed out of a single opening in aerosolized form. This product is useful for dispensing aerosolized medication, but not for the topical application of medicine.

There are also syringes that can apply multiple medicines topically. U.S. Pat. No. 4,359,049 (1982) to Redl et al. teaches a multi-chambered syringe that combines three medications for dispensation. The primary use for Redl's syringe is for the topical application of multiple medications to wounds or to the skin. Redl's invention works similar to other multi-chambered syringes. By depressing a plunger, the multiple medications are forced into a mixing chamber and then the mixed medication is forced out of a narrow opening. This syringe is useful in applying multiple topical treatments, but has a lack of flexibility that is similar to other syringes. Redl's invention can only be used to dispense three medications and cannot be easily reconfigured to disperse more or less than three medications.

### SUMMARY OF THE INVENTION

Accordingly, it is an object of the invention to create syringe that dispenses multiple medications at one time that is suitable for topical application on animals.

It is another object of the invention to create a syringe that is capable of dispensing the exact number of medications needed. For example, if a patient only needs one medication, the syringe can dispense one medication, but if a patient needs four medications, the same syringe is capable of dispensing four medications.

It is another object of the invention to create a syringe versatile enough that it can be configured with a hypodermic needle in addition to hypodermic application.

It is another object of the invention to create a syringe that can be adapted for other purposes such as dentistry or oil and gas industrial uses.

It is another object of the present invention to create a dispenser that is easy for a veterinarian to use.

In a preferred embodiment, the above mentioned objectives are met by a syringe comprising: an applicator with a tip; a chamber that is in fluid communication with the application tip; a reservoir containing housing at least one ampule of medication; said reservoir separated by a port; a press that is configured be received by the reservoir and push the ampules of medication into the chamber through the port; and a plunger for pushing the ampule contents through the application tip. In one mode of operation: ampules of medication (each ampule can contain a different medication) are deposited into the reservoir of the syringe; the press is inserted into the reservoir and manipulated to push the ampules contents through the port into the chamber; and, the plunger is depressed to force the medications through the application tip. The device functions similarly with direct-fill medications (ointments/gels/creams) dispensed from their packaging ‘directly into the reservoir’.

### BRIEF DESCRIPTION OF THE FIGURES

The manner in which these objectives and other desirable characteristics can be obtained is explained in the following description and attached figures in which:

FIG. 1 is a top view of the syringe.

FIG. 2 is an exploded view of the syringe.

FIG. 3 is a close up view of how the ampules fit into the syringe.

FIG. 4 is a cross sectional view of the casing.

These drawings illustrate only typical embodiments of the disclosed subject matter, and are not to be considered limiting of its scope, for the disclosed subject matter may admit to other equally effective embodiments. The figures are not necessarily to scale and certain features and certain views of the figures may be shown exaggerated in scale or in schematic in the interest of clarity and conciseness.

### DETAILED DESCRIPTION OF THE INVENTION

Generally disclosed is a syringe for the application of topical medication. In a preferred embodiment, disclosed a syringe comprising: an applicator with a tip; a chamber that is in fluid communication with the application tip; a reservoir containing housing at least one ampule medication or direct-filled medication; said reservoir separated by a port; a press that is configured be received by the reservoir and push the ampules of medication into the chamber through the port; and a plunger for pushing the ampules' contents of medication through the application tip. In one mode of operation: ampules of medication (each ampule can contain a different medication) are deposited into the reservoir of the syringe; the press is inserted into the reservoir and manipulated to push the ampules' contents through the port into the chamber; and, the plunger is depressed to force the medications through the application tip 4. The more detailed features of the preferred embodiment are disclosed with reference to the figures.

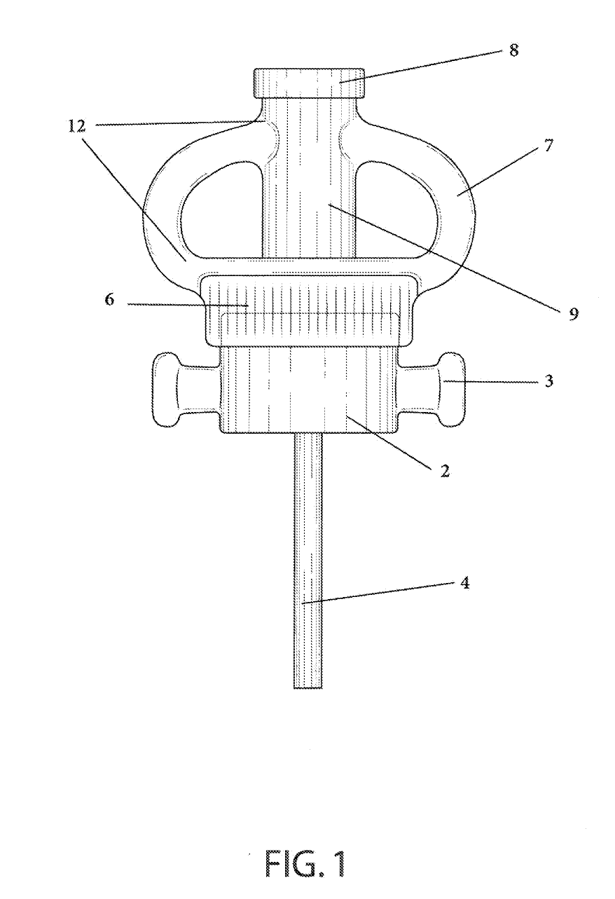

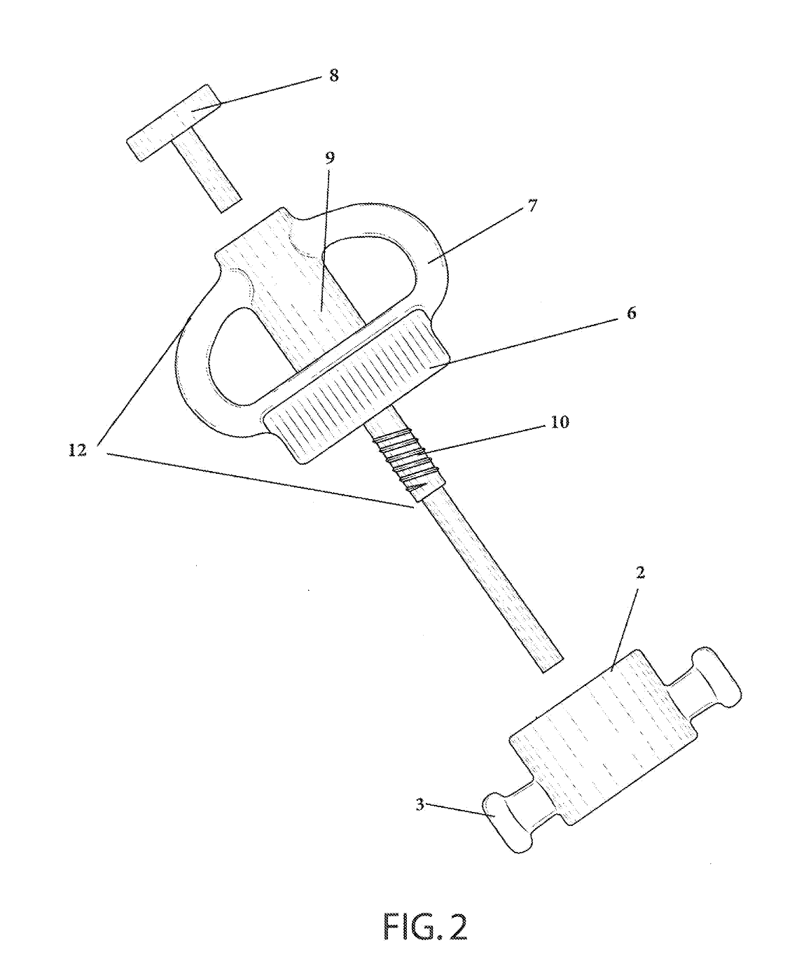

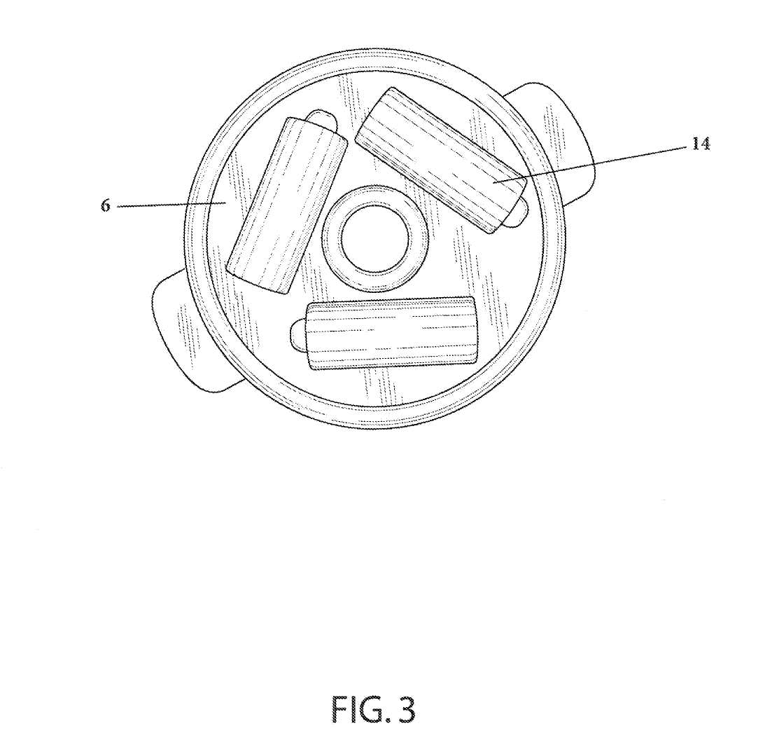

FIG. 1 shows a top view of the assembled syringe 1. FIG. 2 shows an exploded view of the syringe. As shown, components of the depicted syringe 1 are the press 2, the casing 12 (including the application tip 4, the reservoir 6 and the chamber 9), and the plunger 8. In operation, the syringe 1 is configured to receive one or more ampules 14 (shown in FIG. 3, 14) in the reservoir 6. In a preferred embodiment, the ampules 14 may be preferably filled with topical medications.

FIGS. 1 and 2 depict the casing 12 of the syringe 1. In a preferred embodiment, the casing 12 is fluidly connected to the application tip 4. As shown, the application tip 4 protrudes from one end of the circular section where medicine is to be dispensed. As discussed in greater detail below, the basic operation of the syringe is accomplished via depositing a substance into the chamber 9 of the casing 12 and manipulating the plunger 8 to force the substance through the application tip 4 for ejection.

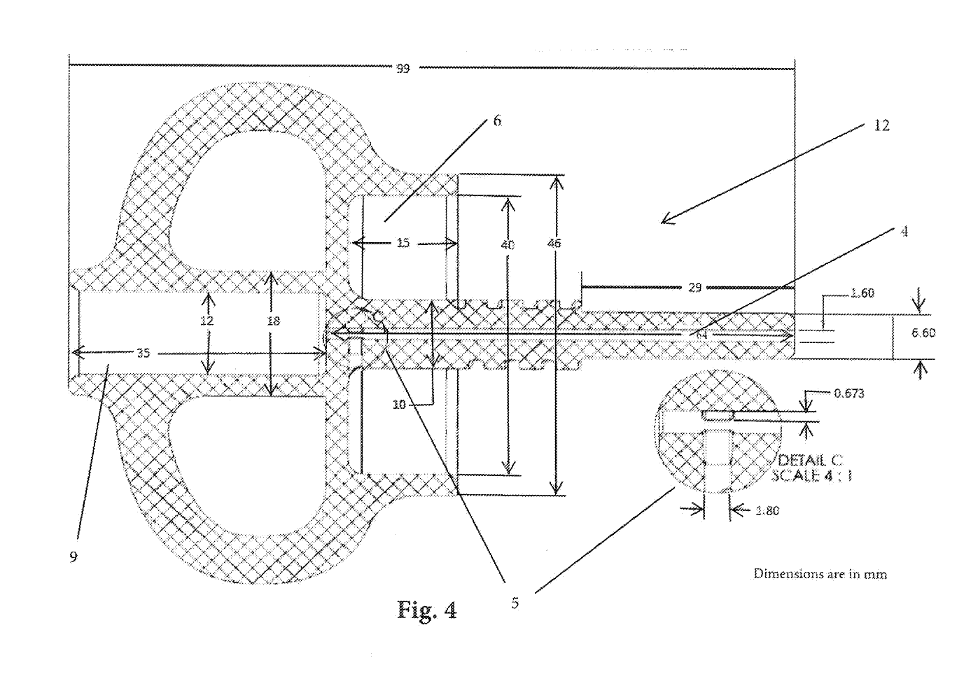

FIGS. 1 and 2 preferably depict the structure of the casing 12. As shown, the casing 12 is generally tubiform. The casing 12 is suitably defined by the chamber 9, the reservoir 6, and the application tip 4. The reservoir 6 and the chamber 9 are separated by a port (see FIG. 4, 5) with the application tip 4 running coaxial to the reservoir and in fluid communication with the chamber. The casing 12 also features two handles 7 which aid in the user in gripping the syringe 1. As discussed in detail below, the reservoir 6 is a hollow area of the syringe where disparate medications can be mixed and pressed through the port 5 into the chamber 9 for application via the tip 4. The casing 12 is preferably made of a plastic or polymer, but it may be made of glass, ceramic, stainless steel or other durable, sanitizable material.

FIGS. 1 and 2 also depict the press 2. As shown, a preferred embodiment of the press 2 is a detachable component from casing 12. The press 2 mixes the various topical treatments by pushing the ampules' contents through the port 5 into the chamber 9. The press 2 a round shape with two grips 3 on the either side. There is a hole in the middle of the press 2 for receiving the application tip 4, which allows the press 2 to fit onto the casing 12 while being inserted into the reservoir 6. The press 2 mechanically interacts with the casing 12 via threading 10 on the application tip 4 and corresponding threading in the hole of the press 2. In one embodiment, turning the press 2 causes it to move up the threading 10. In operation, moving the press 2 to within the reservoir 6 will squeeze the ampules 14 (See FIG. 3, 14) until the contents of the ampules 14, which is preferably medicine, spill into the reservoir 9. As the press 2 moves further up the chamber, the contents of the ampules 14 are pushed through the port 5 into the chamber 9 where they are mixed. The press 2 is preferably made of a strong plastic material that is capable of crushing the ampules 14. It may also be made of metal or porcelain. In some embodiments the top of the press 2 is made of a firm rubber so that contents of the reservoir 6 do not leak at the interface of the reservoir 6 and press 2.

When the press 2 is in the position show in FIG. 1, the highest possible position on the threading 10, the medication from the ampules 14 is ready to be dispensed from the application tip 4. The medication is dispensed in a manner that is common to syringes, e.g., via manipulating the plunger 8. In the preferred embodiment shown in FIG. 2, the plunger 8 is a separate piece that is inserted at the top of the casing 12. When the plunger 8 is inserted into the casing 12 and pressed, it forces the medicine in the chamber 9 into the tip 4. For this purpose, the plunger may include an air-tight interface with the walls of the chamber 9. In the preferred embodiment, the plunger 8 is made of the same material as the casing 12. In some embodiments, the plunger has a rubber coating on the top which aids a user pressing down on the plunger 8. In another embodiment, the plunger may be graduated in order to show the user how much of the medicine has been dispensed.

As discussed above, manipulating the plunger 8 forces the medicine through the tip 4. The tip 4 is connected to the casing 12. The bottom of the tip 4 has an opening so that medication is slowly dispensed in a controlled manner. The tip 4 suitably allows medication to be applied topically to a wound, skin or orifice such as the canine ear canal. The syringe also features a disposable cap 16 that covers the tip (not shown). In an embodiment not shown, the tip can be replaced with a hypodermic needle and the syringe can be used to inject medication into a patient instead of applying medication topically.

FIG. 3 is a bottom view of the reservoir 6 with the ampules 14 loaded inside of the reservoir. The ampules 14 are small packets pre-filled with a liquid, gel, oil, or powder. In the preferred embodiment, the ampules 14 have been pre-sealed using the blow fig seal process. Blow fill seal is a method for preparing sterile pharmaceuticals. The ampules 14 are formed, filled, and sealed in one continuous process in a sterile enclosed area inside of a machine, ensuring the sterility of the medicine inside of the ampule 14. The ampules 14 may also be blister packs or encapsulated medication.

In the preferred embodiment, the ampules 14 are filled with disparate topical medications, but the syringe is versatile enough that it can be used for other functions. For example, the ampules 14 may be filled with epoxies for industrial use or tooth filling materials for dental use. The ampules 14 may be made of a soft plastic material, such as polypropylene and polyethylene, which easily gives way and allows the medication to disperse into the reservoir 6 when pressure is applied in the form of the press 2. Once the contents of the ampules 14 have been dispensed, the ampules 14 should be discarded. As shown in FIG. 3 three ampules 14 are loaded in the reservoir 6. A veterinarian can load fewer ampules 14 or more ampules 14 into the reservoir 6 depending on the needs of the patient. The ampules 14 can be made in smaller sizes, so it would be possible to fit additional ampules 14 in the reservoir 6. The reservoir 6 can also be directly filled with one or ore topical medications, depending on the needs of a patient.

FIG. 4 is a cross-sectional view of the casing 12. This view depicts the location of the port 5 in relation to the tip 4, the reservoir 6, and the chamber 9. The port 5 fluidly connects the reservoir 6 to the tip 4 and the chamber 9. As the press 2 moves further up the chamber, the medicine is pushed through the port 5 into the chamber 9 where they are mixed.

It is to be noted that appended drawings illustrate only typical embodiments of this invention, are not to scale, and therefore are not to be considered limiting of its scope, for the invention may admit to other equally effective embodiments which are appreciated by those skilled in the arts.

All features disclosed in this specification, including any accompanying claims, abstract, and drawing, may be replaced by alternative features serving the same, equivalent or similar purpose, unless expressly stated otherwise. Thus, unless expressly stated otherwise, each feature disclosed is one example only of a generic series of equivalent or similar features.

Any element in a claim that does not explicitly state “means for” performing a specified function, or “step of” in the clause as specified in 35 §112, paragraph 6 may not be intended as a means plus claim.

The invention claimed is:

1. A syringe comprising:
an application tip;
a chamber
a reservoir capable of housing at least one ampule;
a press; and
a plunger.

2. A syringe according to claim 1 where the chamber and the press are threaded.

3. A syringe according to claim 1 where the tip syringe features a cap.

4. A syringe according to claim 1 capable of holding at least three ampules.

5. A syringe according to claim 1 where the tip is a hypodermic needle.

6. A method of dispensing a substance from a syringe comprising;
placing at least one substance to be dispensed into a reservoir of a syringe;
rotating a press into the reservoir so that the press causes the ampules to open in the reservoir;
manipulating the press further, so that the contents of the substance is mixed inside a reservoir;
pushing a plunger into the reservoir, so that the contents of the reservoir are forced out a tip;
applying the tip to a surface.

7. A method according to claim 6 where the substance dispensed is medicine.

8. A method according to claim 7 where the medicine is applied topically.

9. A method according to claim 7 where the medicine is injected into a patient.

10. A method according to claim 7 where the medicine is applied to an orifice.

11. A method according to claim 8 where the medication is applied to the skin of an animal.

12. A method according to claim 8 where the medication is applied to a wound of an animal.

13. A method according to claim 6 where the substance to be dispensed in the form of an ampule.

14. A method according to claim 6 where the substance to be dispensed is in the form of a capsule.

15. A method according to claim 6 where the substance to be dispensed in the form of a blister pack.

16. A syringe according to claim 1 where the plunger is graduated.
